(19) United States
(12) Patent Application Publication
(10) Pub. No.: US 2016/0242667 A1
(43) Pub. Date: Aug. 25, 2016

(54) TISSUE CONTACT SENSING USING A MEDICAL DEVICE

(71) Applicant: Boston Scientific Scimed Inc., Maple Grove, MN (US)

(72) Inventors: Leon Fay, Lexington, MA (US); Paul Hultz, Brookline, NH (US); Doron Harlev, Brookline, NH (US)

(21) Appl. No.: 15/048,497

(22) Filed: Feb. 19, 2016

Related U.S. Application Data
(60) Provisional application No. 62/118,897, filed on Feb. 20, 2015.

(51) Int. Cl. A61B 5/042; A61B 18/14; A61B 18/08; A61B 5/053; A61B 5/00

(52) U.S. Cl. CPC A61B 5/0422; A61B 5/0538; A61B 5/6858; A61B 18/082; A61B 18/14

(57) ABSTRACT

Medical devices and methods for making and using medical devices are disclosed. An example system for sensing tissue contact is disclosed. The system comprises a catheter shaft including a distal end portion. The distal end portion includes a sensing assembly having a plurality of electrodes. The plurality of electrodes includes a current-carrying electrode, a first sensing electrode and a second sensing electrode. The first sensing electrode is positioned a first distance from the current-carrying electrode. The second sensing electrode is positioned a second distance from the current-carrying electrode and the first distance is different from the second distance. The system also includes a controller coupled to the plurality of mapping electrodes. The controller is capable of calculating a parameter based at least in part on the first and the second distances.

### CROSS-REFERENCE TO RELATED APPLICATION

This application claims priority to Provisional Application No. 62/118,897, filed Feb. 20, 2015, which is herein incorporated by reference in its entirety.

### TECHNICAL FIELD

The present disclosure pertains to medical devices, and methods for manufacturing medical devices. More particularly, the present disclosure pertains to tissue diagnosis and/or ablation.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example electrophysiology medical device is disclosed. The medical device comprises:
a catheter shaft including a distal end portion, wherein the distal end portion includes a sensing assembly having a plurality of mapping electrodes;
wherein the plurality of mapping electrodes includes at least one current-carrying electrode, a first sensing electrode and a second sensing electrode;
wherein the first sensing electrode is spaced from the current-carrying electrode a first distance;
wherein the second sensing electrode is spaced from the current-carrying electrode a second distance;
wherein the first distance is different from the second distance; and
a controller coupled to the plurality of mapping electrodes;
wherein the controller is capable of calculating a parameter based at least in part on the first and the second distances.

Alternatively or additionally, the parameter indicates the proximity of the medical device to tissue.

Alternatively or additionally, calculating the parameter includes sensing a first voltage potential between the first electrode and one or more return electrodes, and sensing a second voltage potential between the second electrode and one or more return electrodes.

Alternatively or additionally, calculating the parameter includes solving at least one linear equation, and wherein the at least one linear equation includes the first distance, the second distance, the first voltage and the second voltage.

Alternatively or additionally, the sensing assembly includes a plurality of splines, and wherein the plurality of electrodes are disposed on the plurality of splines.

Alternatively or additionally, the sensing assembly includes a plurality of splines, and wherein the plurality of splines includes an outwardly facing surface, and wherein the plurality of electrodes are disposed on the outwardly facing surface.

Alternatively or additionally, the sensing assembly includes a plurality of splines, and wherein the plurality of splines are arranged in a basket.

Alternatively or additionally, the plurality of electrodes are each designed to sequentially and/or simultaneously operate in a sensing configuration and a current-carrying configuration.

Alternatively or additionally, further comprising displaying the parameter on a display.

Alternatively or additionally, displaying the parameter includes displaying a confidence value corresponding to the parameter.

Alternatively or additionally, the displaying the parameter on a display further includes displaying an anatomical shell and/or an electroanatomical map that indicates the proximity of one or more of the plurality of electrodes to tissue.

Another example system for sensing tissue contact comprises:
a catheter shaft including a distal end portion, wherein the distal end portion includes a sensing assembly having a plurality of electrodes;
wherein the plurality of electrodes includes a current-carrying electrode, a first sensing electrode and a second sensing electrode;
wherein the first sensing electrode is positioned a first distance from the current-carrying electrode;
wherein the second sensing electrode is positioned a second distance from the current-carrying electrode;
wherein the first distance is different from the second distance;
   a processor, wherein the processor is designed to:
      simultaneously detect:
         (a) a first parameter based at least in part on the first and second distances, and
         (b) an impedance increase across at least one of the plurality of electrodes.

Alternatively or additionally, wherein the impedance increase is defined by a change in impedance by at least 100%.

Alternatively or additionally, wherein simultaneously detecting an impedance increase indicates that at least one of the plurality of electrodes is embedded in tissue.

Alternatively or additionally, wherein simultaneously detecting a first parameter based at least in part on the first and second distances includes sensing a first voltage potential between the first electrode and one or more return electrodes, and sensing a second voltage potential between the second electrode and the one or more return electrodes.

Alternatively or additionally, wherein simultaneously detecting a first parameter includes solving at least one linear equation, and wherein the at least one linear equation includes the first distance, the second distance, the first voltage and the second voltage.

Alternatively or additionally, wherein simultaneously detecting an impedance increase includes measuring an impedance between a current-carrying electrode and one or more return electrodes

Another example electrophysiology medical device comprises:
a catheter shaft including a distal end portion;
a sensing assembly having a plurality of electrodes, wherein the plurality of electrodes includes four or more terminals;
wherein the four or more terminals includes one or more current-carrying electrodes and one or more sensing electrodes;
wherein the one or more current-carrying electrodes, the one or more sensing electrodes, or both includes a mapping electrode;
wherein the four or more terminals are designed to measure an electrical characteristic; and
a processor coupled to the sensing assembly.

Alternatively or additionally, wherein the electrical characteristic is a voltage, an impedance, or both.

Alternatively or additionally, wherein the electrical characteristic indicates the proximity of the medical device to tissue.

Another medical device for sensing contact with tissue comprises:
a catheter shaft, wherein the shaft includes a distal portion;
a sensing assembly coupled to the distal portion of the catheter shaft, wherein the sensing assembly includes a plurality of electrodes; and
wherein the plurality of electrodes includes at least a first mapping electrode, and wherein the first mapping electrode is designed to detect an impedance increase, and wherein the impedance increase is defined by an increase of an impedance by 100% or more.

The above summary of some embodiments is not intended to describe each disclosed embodiment or every implementation of the present disclosure. The Figures, and Detailed Description, which follow, more particularly exemplify these embodiments.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The disclosure may be more completely understood in consideration of the following detailed description in connection with the accompanying drawings, in which:

FIG. 1 is a plan view of an example tissue diagnosis and/or ablation system;

FIG. 2 illustrates an example medical device including an electrode structure, a catheter shaft and a handle;

FIG. 3 illustrates an example basket electrode structure including sensing electrodes;

FIG. 4 illustrates an example electrode having multiple layers;

FIG. 5 illustrates an example electrode having multiple layers;

FIGS. 6-8 illustrate an example electrode structure utilized with the system of FIG. 1 moving between blood and tissue;

FIG. 9 illustrates an example electrode structure having multiple sensing electrodes spaced different distances away from a tip electrode.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to an embodiment“, some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Cardiac arrhythmia and/or other cardiac pathology contributing to abnormal heart function may originate in cardiac cellular tissue. One technique that may be utilized to treat the arrhythmia and/or cardiac pathology may include ablation of tissue substrates contributing to the arrhythmia and/or cardiac pathology. Ablation by heat, chemicals or other means of creating a lesion in the tissue substrate may isolate diseased tissue from normal heart circuits. In some instances, electrophysiology therapy may involve locating tissue contributing to the arrhythmia and/or cardiac pathology using a mapping and/or diagnosing catheter and then using an ablation electrode to destroy and/or isolate the diseased tissue.

Prior to performing an ablation procedure, a physician and/or clinician may utilize specialized mapping and/or diagnostic catheters to precisely locate tissue contributing and/or causing an arrhythmia or other cardiac pathology. It is often desirable to precisely locate the targeted tissue prior to performing an ablation procedure in order to effectively alleviate and/or eliminate the arrhythmia and/or cardiac pathology. Further, precise targeting of the tissue may prevent or reduce the likelihood that healthy tissue (located proximate the targeted tissue) is damaged.

Several methods and/or techniques may be employed to precisely locate targeted tissue where an ablation or other therapeutic procedure may be performed. An example method may include utilizing an ablation, mapping and/or diagnostic catheter to determine how close the catheter is to targeted tissue. Further, the ablation, mapping and/or diagnostic catheter may include one or more sensing electrodes located on a distal portion of the catheter. The electrodes may sense, measure and/or provide a processor with information relating to electrical characteristics of the cardiac tissue and surrounding media. Using the sensed and/or measured information, the processor may be able to correlate the spatial location of the distal portion of the catheter to the cardiac tissue. For example, electrodes may sense the impedance, resistance, voltage potential, etc. of the cardiac tissue and/or surrounding media and determine how far a distal portion of a diagnostic and/or ablation catheter is to cardiac tissue.

In general, the size, shape and spacing of electrodes on a diagnostic (e.g. mapping) catheter may contribute to the accuracy to which a diagnostic catheter may sense and/or measure electrical characteristics. For example, some methods and/or techniques disclosed herein may emit a current from a first electrode and measure a voltage, impedance or other electrical characteristic of local tissue using other electrodes. Further, in some instances the size of an electrode may directly influence the magnitude of the measured response by a processor. For example, as will be discussed in detail later, impedance measurements corresponding to tissue contact may be magnified by using small, flat electrodes as compared to other sensing electrode configurations. Small, flat electrodes may increase the likelihood that a given electrode may become fully embedded and/or surrounded in cardiac tissue. Fully embedding a sensing electrode within cardiac tissue may directly correspond to determining whether the electrode is in contact with the cardiac tissue.

In addition, larger electrodes may be more susceptible (as compared to smaller electrodes) to detecting far field electrical activity. Detection of far field electrical activity may negatively affect the detection of local (e.g. targeted) electrical activity.

Therefore, in some instances it may be desirable to utilize and incorporate small, flat electrodes into the distal portion of a mapping and/or a diagnostic catheter. For example, some of the medical devices and methods disclosed herein may include sensing and measuring electrical activity using one or more relatively small, flat electrodes in conjunction with other sensing methods, electrodes, ablation electrodes, diagnostic catheters and/or other medical devices. Further, some of the medical devices and methods disclosed herein may utilize electrical characteristics collected from small, flat electrodes to assess tissue proximity and/or contact. Other methods and medical devices are also disclosed.

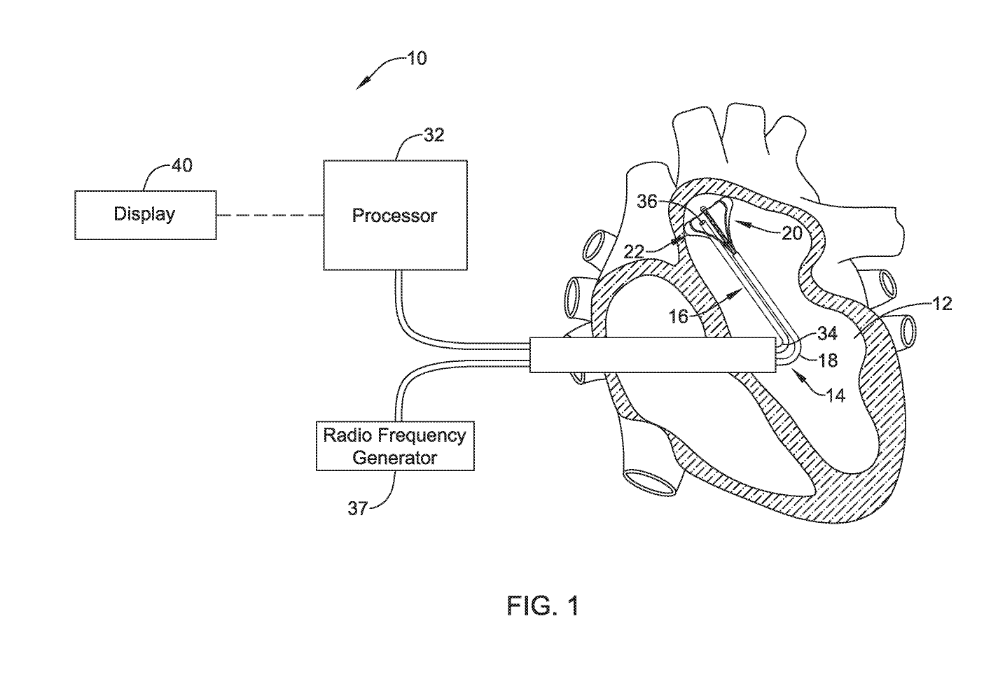

FIG. 1 is a schematic view of a system 10 for accessing a targeted tissue region in the body of a patient for diagnostic and/or therapeutic purposes. FIG. 1 generally shows the system 10 deployed in a region of the heart. For example, system 10 may be deployed in any chamber of the heart, such as the left atrium, left ventricle, right atrium, or right ventricle, another region of the cardiovascular system, or other anatomical region. While the illustrated embodiment shows the system 10 being used for sensing contact and/or proximity to myocardial tissue, the system 10 (and the methods described herein) may alternatively be configured for use in other tissue applications, such as procedures for sensing tissue in the prostate, brain, gall bladder, uterus, nerves, blood vessels and other regions of the body, including body regions not typically accessed by a catheter.

System 10 includes a mapping catheter or probe 14. In some instances, system 10 may also include an ablation catheter or probe 16. Each probe 14/16 may be separately introduced into the selected heart region 12 through a vein or artery (e.g., the femoral vein or artery) using a suitable percutaneous access technique. Alternatively, mapping probe 14 and ablation probe 16 can be assembled in an integrated structure for simultaneous introduction and deployment in the heart region 12.

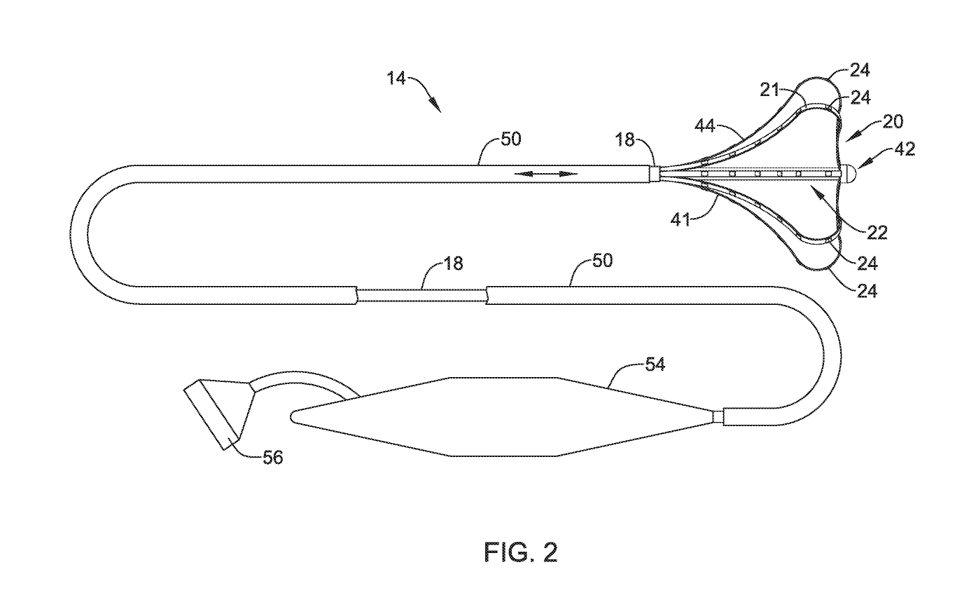

Mapping probe 14 may include flexible catheter body 18. The distal end of catheter body 18 carries three-dimensional multiple electrode structure 20. In the illustrated embodiment, structure 20 takes the form of a basket defining an open interior space 22 (see FIG. 2), although other multiple electrode structures could be used. Structure 20 carries a plurality of mapping electrodes 24 (not explicitly shown on FIG. 1, but shown on FIG. 2) each having an electrode location on structure 20 and a conductive member. Each mapping electrode 24 may be configured to sense electrical characteristics (e.g. voltage and/or impedance) in an adjacent anatomical region.

Electrodes 24 may be electrically coupled to processing system 32. A signal wire (not shown) may be electrically coupled to each electrode 24 on structure 20. The signal wires may extend through body 18 of probe 14 and electrically couple each electrode 24 to an input of processing system 32. Electrodes 24 may sense electrical characteristics correlated to an anatomical region adjacent to their physical location within the heart. The sensed cardiac electrical characteristic (e.g., voltage, impedance, etc.) may be processed by processing system 32 to assist a user, for example a physician, by generating processed output—e.g. an anatomical map (e.g., 3D map of heart chamber)—to identify one or more sites within the heart appropriate for a diagnostic and/or treatment procedure, such as an ablation procedure.

Processing system 32 may include dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired physiological activity. In some examples, processing system 32 may include a general purpose microprocessor and/or a specialized microprocessor (e.g., a digital signal processor, or DSP, which may be optimized for processing activation signals) that executes instructions to receive, analyze and display information associated with the received physiological activity. In such examples, processing system 32 can include program instructions, which when executed, perform part of the signal processing. Program instructions can include, for example, firmware, microcode or application code that is executed by microprocessors or microcontrollers. The above-mentioned implementations are merely exemplary, and the reader will appreciate that processing system 32 can take any suitable form for receiving electrical signals and processing the received electrical signals.

Ablation probe 16 may include flexible catheter body 34 that carries one or more ablation electrodes 36. The one or more ablation electrodes 36 may be electrically connected to radio frequency (RF) generator 37 that is configured to deliver ablation energy to the one or more ablation electrodes 36. Ablation probe 16 may be movable with respect to the anatomical feature to be treated, as well as structure 20. Ablation probe 16 may be positionable between or adjacent to mapping electrodes 24 of structure 20 as the one or more ablation electrodes 36 are positioned with respect to the tissue to be treated.

Processing system 32 may output data to a suitable device, for example display device 40, which may display relevant information for a user. In some examples, device 40 is a display (e.g. a CRT, LED), or other type of display, or a printer. Device 40 may present the relevant characteristics in a format useful to the user. In addition, processing system 32 may generate position-identifying output for display on device 40 that aids the user in guiding an ablation electrode into contact with tissue at the site identified for ablation.

FIG. 2 illustrates mapping catheter 14 and shows mapping electrodes 24 at the distal end suitable for use in system 10 shown in FIG. 1. Mapping catheter 14 may include flexible catheter body 18, the distal end of which may carry three-dimensional multiple electrode structure 20 with mapping electrodes or sensors 24. Mapping electrodes 24 may sense electrical characteristics (e.g. voltage, impedance) in the myocardial tissue. The sensed cardiac electrical activity may be processed by the processing system 32 to assist a user in identifying the site or sites having a heart rhythm disorder or other myocardial pathology via generated and displayed relevant characteristics. This information can then be used to determine an appropriate location for applying appropriate therapy, such as ablation, to the identified sites, and to navigate the one or more ablation electrodes 36 to the identified sites.

Multiple electrode structure 20 may include base member 41 and distal tip 42 between which flexible splines 44 generally extend in a circumferentially spaced relationship. As discussed herein, structure 20 may take the form of a basket defining an open interior space 22. Structure 20 may flare distally from a constrained configuration to a more open configuration. In some examples, the splines 44 are made of a resilient inert material, such as Nitinol, other metals, silicone rubber, suitable polymers, or the like and are connected between base member 41 and distal tip 42. In some instances, splines 44 may be made of parylene. As shown in FIG. 2, splines 44 may include a substantially flat outwardly facing surface 21 and may resemble strips having a substantially reduced thickness and extending from distal tip 42 to catheter body 18. In some instances, splines 44 may have a rectangular and/or ovular cross-section. These are just examples; other cross-sectional shapes are contemplated. Other shapes, configurations and arrangements are contemplated including arrangements disclosed in U.S. Pat. No. 8,103,327, the entire disclosure of which is herein incorporated by reference.

In some embodiments described herein, distal tip 42 may include an ablation electrode. Further, in some instances distal tip 42 may include an ablation electrode coupled to RF generator 37. Distal tip 42 may emit ablative energy and/or an electrical current.

In some instances, splines 44 are positioned in a resilient, pretensioned condition, to bend and conform to the tissue surface they contact. In the example illustrated in FIG. 2, eight splines 44 form three-dimensional multiple electrode structure 20. Additional or fewer splines 44 could be used in other examples. As illustrated, each spline 44 carries eight mapping electrodes 24. Additional or fewer mapping electrodes 24 could be disposed on each spline 44 in other examples of three dimensional multiple electrode structure 20. Slidable sheath 50 may be movable along the major axis of catheter body 18. Moving sheath 50 distally relative to catheter body 18 may cause sheath 50 to move over structure 20, thereby collapsing structure 20 into a compact, low profile condition suitable for introduction into and/or removal from an interior space of an anatomical structure, such as, for example, the heart. In contrast, moving sheath 50 proximally relative to the catheter body may expose structure 20, allowing structure 20 to elastically expand and assume the pre-tensioned position illustrated in FIG. 2.

In other examples, slidable sheath 50 (or other deployment shaft) may be connected to distal tip 42. Further, deployment of structure 20 may include manipulating a slidable sheath 50 (or other deployment shaft) coupled to distal tip 42. For example, deployment of structure 20 may be accomplished by pulling slidable sheath 50 (or other deployment shaft) in a proximal direction. The proximal movement of slidable sheath 50 (or other deployment shaft) may result in distal tip 42 moving in a proximal direction. As distal tip 42 moves proximally, it may force splines 44 to flare out and assume the shape of structure 20 shown in FIG. 2, for example.

A signal wire (not shown) may be electrically coupled to each mapping electrode 24. The signal wires may extend through body 18 of mapping catheter 14 (or otherwise through and/or along body 18) into handle 54, in which they are coupled to external connector 56, which may be a multiple pin connector. Connector 56 may electrically couple mapping electrodes 24 to processing system 32. It should be understood that these descriptions are just examples. Some addition details regarding these and other example mapping systems and methods for processing signals generated by a mapping catheter can be found in U.S. Pat. Nos. 6,070,094, 6,233,491, and 6,735,465, the disclosures of which are hereby expressly incorporated herein by reference.

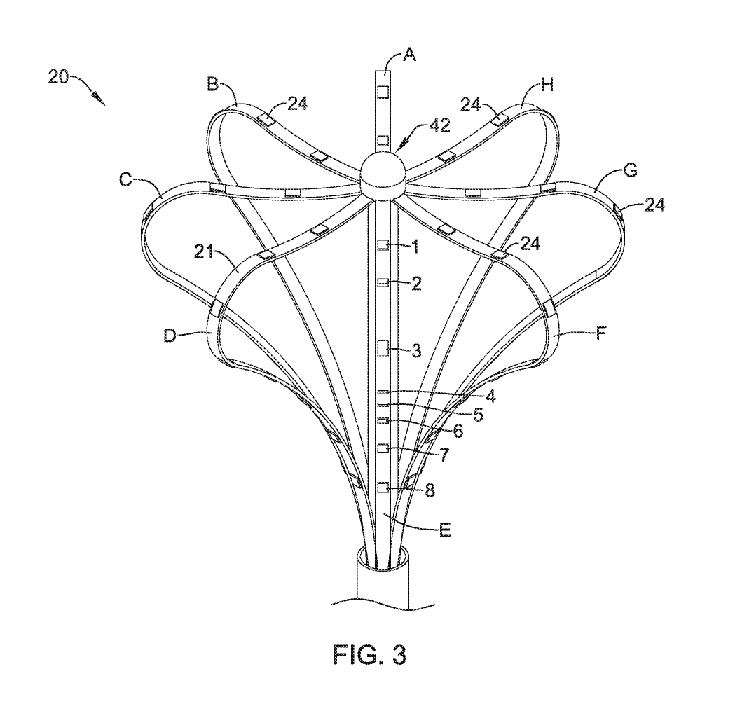

To illustrate the operation of system 10, FIG. 3 is a schematic side view of example basket structure 20 including a plurality of mapping electrodes 24. In the illustrated example, the basket structure includes 64 mapping electrodes 24. Mapping electrodes 24 are disposed in groups of eight electrodes (labeled 1, 2, 3, 4, 5, 6, 7, and 8) on each of eight splines (labeled A, B, C, D, E, F, G, and H). While an arrangement of sixty-four mapping electrodes 24 is shown disposed on basket structure 20, mapping electrodes 24 may alternatively be arranged in different numbers (more or fewer splines and/or electrodes), on different structures, and/or in different positions. In addition, multiple basket structures can be deployed in the same or different anatomical structures to simultaneously obtain signals from different anatomical structures.

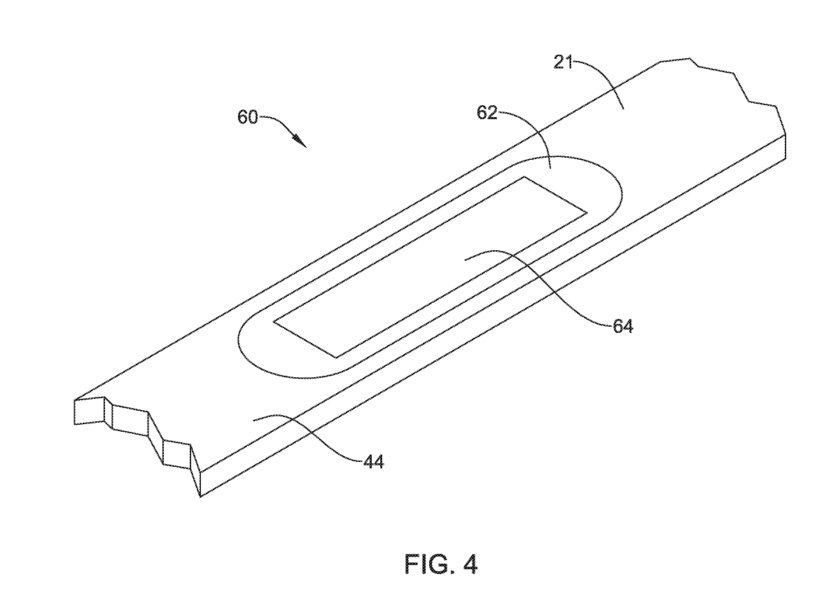

FIG. 4 shows example electrode 60 disposed along spline 44. Electrode 60 may be one of the plurality of mapping electrodes 24. In some instances, such as that shown in FIG. 4, electrode 60 may be affixed along a surface of spline 44. However, it is contemplated that electrode 60 may be coupled to spline 44 using a variety of methodologies. As discussed herein, electrode 60 may be described as being “affixed,” “on” and/or otherwise embedded and/or encased on any structure contemplated herein. This is not intended to be limiting. Positioning/locating electrode 60 along spline 44 may include embedding, partially embedding, encasing, partially encasing, isolating, attaching, affixing, fastening, bonding to the outer surface, embedding within the wall, or the like. Additionally, as shown and described with respect to FIGS. 1-3, it is contemplated that more than one electrode 60 may be affixed to spline 44.

In some instances, electrode 60 may include base layer 62 and top layer 64. Top layer 64 may be a layer of material applied over base layer 62. For example, in some instances base layer 62 may be made from gold, while top layer 64 may be made of iridium oxide. A masked layer of parylene may be applied over base layer 62 such that only top layer 64 is exposed. In some applications, base layer 62 may be applied as a plated layer. For example, electrode structure 20 may be constructed from a method of manufacturing that may bear some resemblance to an analogous processes utilized in the manufacturing of semiconductors. In other words, the manufacturing process may include “printing” or “layering” top layer 64 along, atop, within, embedded with, etc. bottom layer 62. Further, the example method of manufacturing may include forming bottom layer 62 of material (e.g. gold) upon which top layer 64 (e.g. iridium oxide) may be “printed,” “layered,” “plated,” “sputtered,” or the like. The manufacturing method may further include layering one or more additional layers on top and/or within the either top layer 64 and/or bottom layer 62. Additional layers of material may include traces, circuit components, or the like. In some instances, a portion of a layer may be removed to expose an underlying layer. These are just examples, other materials and manufacturing techniques are contemplated. Further, while the following discussion is directed toward the electrode structure previously described, it is contemplated that a variety of electrode designs, including those without multiple layers, may be utilized with any of the medical devices, systems or methodologies disclosed herein.

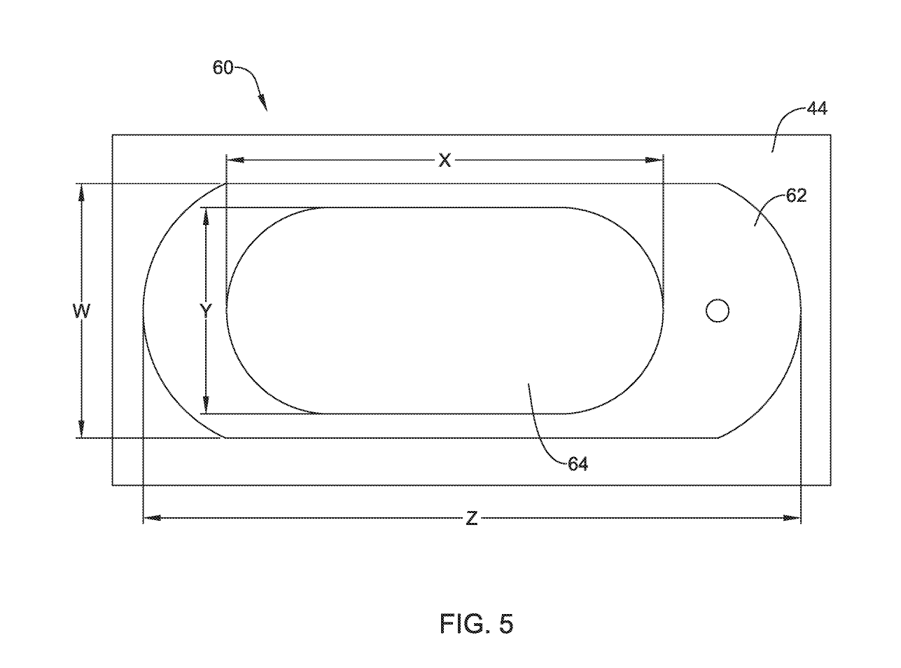

FIG. 5 shows a plan view of electrode 60 including spline 44, bottom layer 62 and top layer 64. FIG. 5 shows bottom layer 62 beneath top layer 64 and having a length substantially aligned with the length of spline 44. The length of top layer 64 is depicted by the letter “X.” Further, FIG. 5 shows top layer 64 having a width perpendicular to the longitudinal axis of spline 44 and depicted by the letter “Y.” In some instances, top layer 64 may have an exposed length of 0.25-1.5 mm, 0.5-1.25 mm, 0.75-1.0 mm, or the like. In some instances, the length of top layer 64 may be 0.95 mm.

As shown in FIGS. 4 & 5, electrode 60 may have a substantially low profile. This reduced profile may allow electrode 60 to be embedded within spline 44, set “flush” with the exterior surface 21 of spline 44, sit slightly “proud” of the top surface of spline 44 or sit significantly proud of spline 44. In instances where electrode 60 is embedded within spline 44, surfaces of electrode 60 other than top layer 64 may not be exposed to surfaces in contact with the outermost surface of spline 44. In other words, in some cases the only exposed surfaces of electrode 60 include top layer 64.

FIGS. 4 & 5 depict electrode 60 (including bottom layer 62 and top layer 64) as having generally rectangular shapes. This is merely an example. It is contemplated that electrode 60 (and any portion thereof) may be circular, trapezoidal, square, oval, triangular, or the like.

As stated above, basket structure 20 may be advanced into an anatomical structure and positioned adjacent to the anatomical structure to be treated (e.g. left atrium, left ventricle, right atrium, or right ventricle of the heart). Additionally, processing system 32 may be configured to record selected electrical characteristics (e.g. voltage, impedance, etc.) from each mapping electrode 24. In some instances, these electrical characteristics may provide diagnostic information corresponding to the relationship between the basket structure 20 and the anatomical structure.

An example method for assessing tissue contact may include determining a parameter of a model and observing changes in the parameter as the distal end of catheter 14 moves between different mediums (e.g. as between blood and tissue). It can be appreciated that catheter 14 may move between blood and tissue as catheter 14 is manipulated within a cardiac chamber.

A scaling factor may be a parameter in a model used for this purpose. The model may relate to one or more potential differences between one or more sensing electrodes and a reference electrode. A reference electrode may be an electrode placed a distance away from the potential measuring electrodes. For example, a reference electrode may be placed on the back of a patient. Sensing electrodes may be one of several combinations of electrodes 24 on basket structure 20.

Additionally or alternatively, the model may also relate to the distance in space between a current-carrying electrode and one or more sensing electrodes. The current-carrying electrode may take a variety of forms. For example, the current-carrying electrode may be any one of mapping electrodes 24 on basket structure 20 and/or a distal ablation tip electrode located on distal tip 42.

In some configurations, the potential measurement between a sensing electrode and a reference electrode may be modeled as being inversely proportional to the distance between a current-carrying electrode and a sensing electrode. For example, the relationship may be modeled as:

$${ϕ}_{S \text{?}} = \frac{K}{\vert {γ}_{CCE 1} - {γ}_{SE 2} \vert} + C$$

$$\text{?} \text{indicates text missing or illegible when filed}$$

In this example, the parameter K may be used to assess tissue contact. The above equation is just an example. Other models and parameters are contemplated. In some instances, the parameter K may be referred to as a “K-factor.”

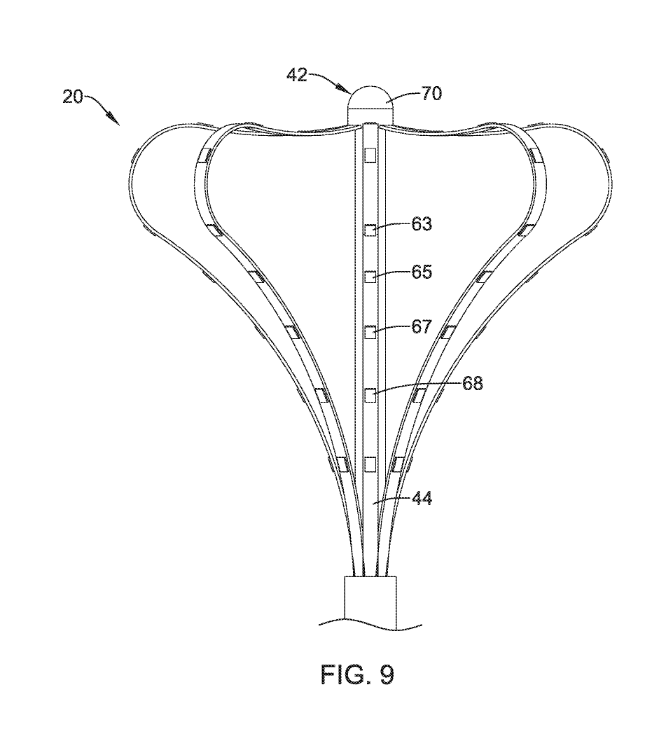

As stated above, the model may relate to both the potential differences between one or more sensing electrodes and the distance between a current-carrying electrode and sensing electrodes. For example, FIG. 9 illustrates an example distal tip 42 including a current-carrying electrode 70 and four sensing electrodes 63, 65, 67 and 68. FIG. 9 is just an example. It is understood that combinations and configurations of any of mapping electrodes 24 on electrode structure 20 may be utilized for any embodiment described herein. For example, any one of mapping electrodes 24 may be configured as either a sensing and/or current-carrying electrode.

In some instances, the relationship between the above electrodes and potential values may be represented by the following equation:

$$[ \begin{matrix}{ϕ}_{SE 1} \\ {ϕ}_{SE 2} \\ {ϕ}_{SE 3} \\ {ϕ}_{SE 4}\end{matrix} ] = [ \begin{matrix}\frac{1}{‖ {γ}_{CCE 1} - {γ}_{SE 1} ‖} 1 \\ \frac{1}{‖ {γ}_{CCE 1} - {γ}_{SE 2} ‖} 1 \\ \frac{1}{‖ {γ}_{CCE 1} - {γ}_{SE3} ‖} 1 \\ \frac{1}{‖ {γ}_{CCE 1} - {γ}_{SE 4} ‖} 1\end{matrix} ] [ \begin{matrix}K & C\end{matrix} ]$$

It can be appreciated that the variables

$$[ \begin{matrix}{ϕ}_{SE 1} \\ {ϕ}_{SE 2} \\ {ϕ}_{SE 3} \\ {ϕ}_{SE 4}\end{matrix} ]$$

represent the measured potential difference between the four sensing electrodes (e.g. 63, 65, 67, 68 in FIG. 9) and a reference electrode (not shown in FIG. 9). Additionally, the potential differences may be determined by system 10. Further, it can be appreciated that |r_{CCS1}−r_{SS1}|, |r_{CCS1}−r_{SS2}|, |r_{CCS1}−r_{SS3}| and |r_{CCS1}−r_{SS4}| represent the absolute value of the distance (in space) between the current-carrying electrode (e.g. 70 in FIG. 9) and the four sensing electrodes (e.g. 63, 65, 67, 68 in FIG. 9), respectively. It is further understood that these distances may be determined as the position (and distance) for every sensing electrode in relation to the current-carrying electrode is known. For example, because the electrodes are fixed along the spline, the distance between electrodes on the spline is known. Furthermore, it is contemplated that when the spline is in a non-linear configuration (e.g. expanded), the distance between electrodes can be determined using curvilinear and/or straight line calculation. In other words, the position, and therefore, the distances, between example sensing electrodes 63, 65, 67, 68 and current-carrying electrode 70 are known on electrode structure 20.

The parameters K and C in the above system of linear equations can be estimated using a number of well-known techniques for optimization or linear regression. For example, least squares can be used to estimate K and C. Other methods are contemplated. Furthermore, it can be appreciated that the above system of linear equations may be arranged in other ways. For example, the linear equations may be combined such that the parameter C vanishes and only K remains to be estimated.

Scaling factor K may be inversely proportional to the conductivity of a given medium. In other words, the scaling factor K will be different for two mediums having different conductivities. For example, the conductivity of blood is greater than that of cardiac tissue, and therefore, the scaling factor K will be lower for blood as compared to cardiac tissue.

Knowing the potential differences and absolute distance values, it may be possible to solve the linear equation set (above) for the scaling factor, K. Is should be noted that in order to solve the disclosed linear equation set, sensing electrodes must be located at different distances away from the current injecting electrode. If, for example, the distances were all identical, then the matrix on the right-hand side of the equation would be singular and result in an infinite number of equally valid solutions. Referring to FIG. 9, it can be seen that sensing electrodes 63, 65, 67, 68 are located at different distances from current injecting electrode 70.

FIG. 9 illustrates the sensing electrodes 63, 65, 67, 68 positioned longitudinally along spline 44. However, it is contemplated that the sensing electrodes 63, 65, 67, 68 may be positioned in a configuration other than along the longitudinal axis and yet still maintain variable distances between the sensing electrodes and the current-carrying electrode 70. Additionally, in some instances it may be possible to reduce the number of sensing electrodes to two or three and solve the corresponding linear equation set for scaling factor K. In other instances, it may be desirable to increase the number of sensing electrodes; the parameter K can still be estimated using well-known techniques such as least squares.

It can be appreciated from the above discussion that it may be possible to utilize known variables to solve the disclosed linear equation for the scaling factor K. Therefore, system 10 may determine and compare different scaling factor values as the distal end portion of catheter 14 is moved between different mediums (e.g. blood, tissue). The difference in the scaling factors may be utilized as a diagnostic indicator of tissue contact.

Furthermore, because each individual mapping electrode 24 may be configured as either a sensing and/or current-carrying electrode, more than one electrode may be utilized to indicate tissue contact through the use of multiplexed measurements. Multiplexing may include any of a number of known techniques such as time-division, frequency-division, or code-division multiplexing. For example, in one frequency or time “slot”, electrode 63 may be the current-carrying electrode, while electrodes 65, 67, and 68 may be sensing electrodes. In a second frequency or time slot, electrode 65 may be the current-carrying electrode, while electrodes 63, 67, and 68 may be sensing electrodes. It is understood than any combination of electrodes on structure 20 may be current-carrying and/or the sensing electrodes. Further, because most of the impedance “seen” by the current-carrying electrode is due to the conductive medium nearest the electrode, any given electrode may be indicative of the contact of a different part of the electrode structure 20 with tissue. Multiple electrodes may therefore be combined to provide two or more spatially-distinct contact indicators.

It can be appreciated from the above discussion that the size and arrangement of the mapping electrodes 24 disclosed herein may be more desirable for detecting a localized scaling factor K as compared to other electrode structures. The small, flat electrode geometry may make the applied current distribution more localized to nearby tissue than would be achieved with a larger, non-flat electrode. The close spacing of the mapping electrodes 24 may result in a more localized estimate of the scaling factor than would be achieved with larger electrode spacing.

Using the scaling factor K to assess tissue contact may be highly reliable. However, in some instances, the positioning and/or configuration of system 10 may alter the scaling K-factor results. In these instances, it may be desirable to utilize a supplemental method for assessing tissue contact. A variety of supplemental methods for assessing tissue contact are contemplated. For example, a supplemental method for assessing tissue contact may include comparing the amplitude of measured cardiac activation, or a spatial or temporal derivative thereof, to a threshold value. Another example supplemental method for assessing tissue contact may include determining a threshold impedance value that positively identifies tissue contact. More specifically, in some instances system 10 may be capable of sensing and/or measuring an impedance increase and correlating the impedance increase to a visual, audible, etc. indication of tissue contact.

For example, system 10 may be capable of utilizing threshold impedance measurements to sense contact between mapping electrodes 24 and adjacent tissue. In general, the impedance of a given medium may be measured by applying a known voltage or current to a given medium and measuring the resulting voltage or current. In other words, impedance measurements of a given medium can be obtained by injecting current between two electrodes and measuring the resulting voltage between the same electrodes through which the current was injected. The ratio of the voltage potential provides an indication of the impedance of the medium through which the current traveled.

For example, in some instances a current may be injected between an electrode 24 and one or more return electrodes (e.g. patch electrode, mini-electrode, measuring electrode, sensing electrode, or the like). Impedance of the medium (e.g. tissue, blood) adjacent to a current-carrying electrode 24 may be measured according to the methodology disclosed above. For example, if electrode 24 is adjacent to or embedded in cardiac tissue, the impedance of the cardiac tissue may be determined by measuring the ratio of the voltage potential between electrode 24 and the one or more return electrodes. While the above discussion generally describes utilizing the current carrying electrodes and the return electrode(s) in unipolar mode, it is contemplated that electrodes 24 may be capable of operating, or configured to operate, in bipolar sensing modes.

The size and shape of electrodes 24 may influence the ability (or inability) of electrodes 24 to measure the electrical characteristics (e.g. impedance) of cellular tissue and/or a surrounding medium (e.g. blood). In some instances, the degree of contact that an electrode 24 maintains with the cardiac tissue may influence the magnitude of a sensed electrical response. For example, an exaggerated impedance value may be sensed when electrode 24 is completely covered and/or embedded in tissue. In some instances, this exaggerated impedance value may be described as an “impedance increase.” This impedance increase may, therefore, directly correspond to tissue contact. It can be appreciated that the substantially flat, reduced-profile and relatively smaller shape of electrode 60 shown in FIG. 4 may increase the likelihood that as electrode 60 is positioned adjacent tissue it will be completely covered by tissue and thereby trigger an impedance increase. Further, this impedance increase may be sensed by processing system 32, and in some instances, output a signal to display 40 indicating that electrode 60 has made contact with tissue. The impedance increase may be 100%, 150%, 200%, 250%, 300%, 350%, 400%, 500%, 600%, 700%, 800%, 900%, 1000%, 2000%, 50,000% or more of the magnitude of a measured baseline impedance value.

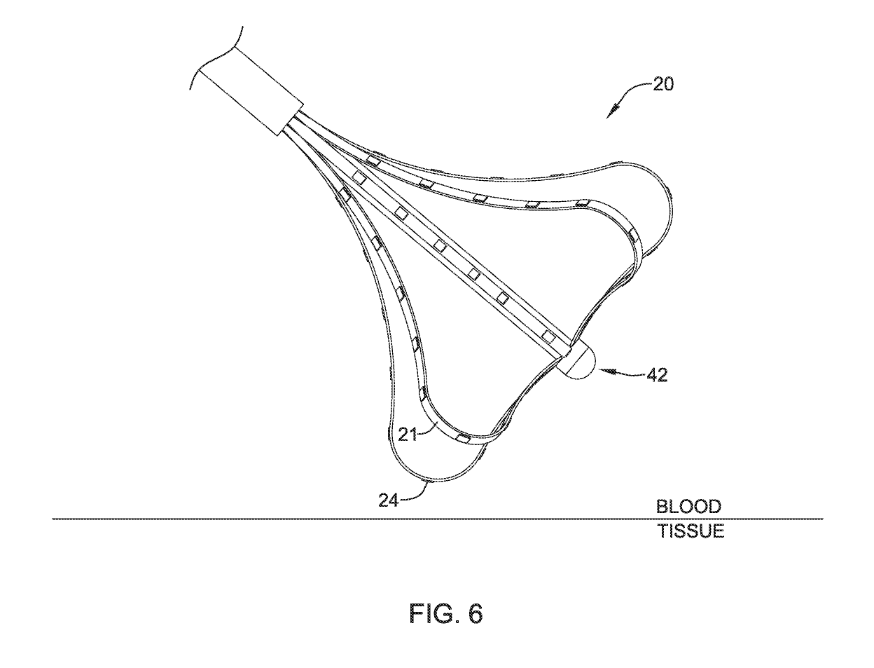

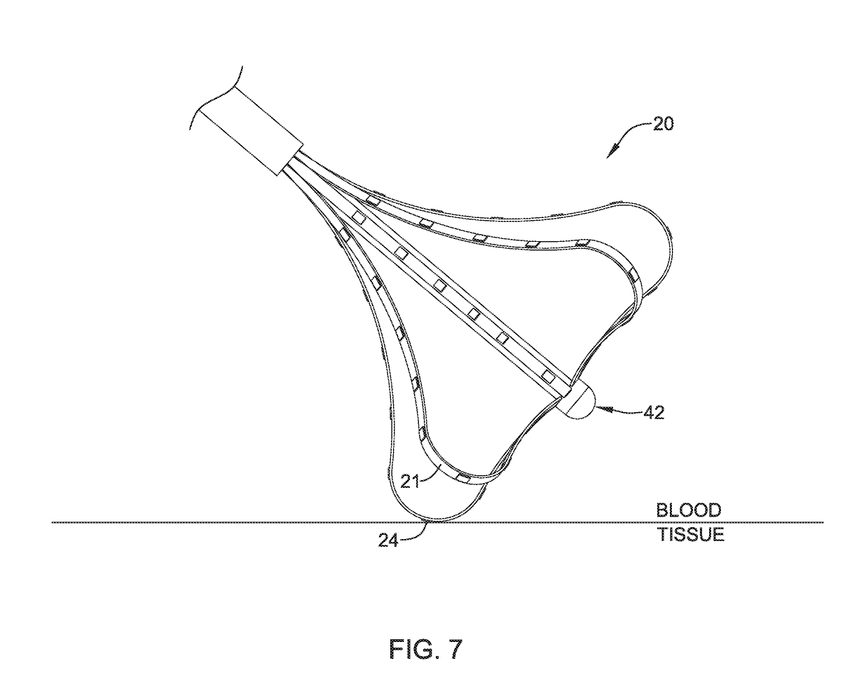

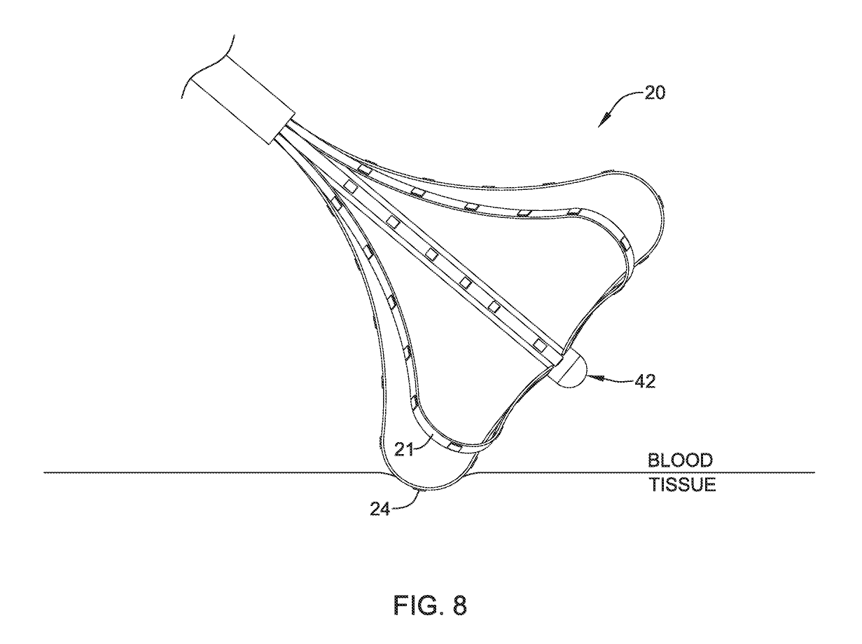

FIGS. 6-8 are a series of drawings that illustrate electrode structure 20 being manipulated within an example cardiac chamber. More specifically, FIGS. 6-8 depict electrode structure 20 advancing through blood toward cardiac tissue. For example, FIG. 6 shows electrode structure 20, including mapping electrode 24, surrounded entirely by blood. FIG. 7 shows mapping electrode 24 positioned at a blood/tissue interface, while FIG. 8 shows electrode structure 20 embedded within cardiac tissue. In these examples, one or more of the plurality of mapping electrodes 24 may be continuously sensing impedance values adjacent to their respective outer surfaces as electrode structure 20 is manipulated within the cardiac chamber. Additionally, processing system 32 may be continuously operating to “sense” an impedance increase from any one of electrodes 24. For example, as mapping electrode 24 moves from a position illustrated in FIG. 6 to an embedded positioned illustrated in FIG. 8, processing system 32 may sense an impedance increase and output a corresponding indication of tissue contact to display 40.

It can be appreciated from the above discussion that the size and shape of the electrodes disclosed herein may be more desirable for detecting an impedance increase as compared to relatively larger, non-flat electrodes. In other words, the electrode size and shape disclosed herein may be more easily covered and/or embedded in adjacent tissue, thereby leading to a greater number of sensed impedance increases and correspondingly positive indications of tissue contact.

In addition or alternatively to any of the embodiments disclosed herein, in some instances it may be desirable to sense tissue contact by simultaneously using two or more methods discussed herein. As stated above, in some instances processing system 32 may have difficulty sensing and comparing a change in K-factor values while being manipulated in an anatomical structure (e.g. cardiac chamber). Therefore, it may be desirable for processing system 32 to sense an impedance increase while simultaneously monitoring and determining changes in the K-factor. However, in some instances processing system 32 may detect an impedance increase correlating to positive tissue contact despite not having sensed tissue contact utilizing the K-factor method. Having detected an impedance increase (in the absence of a positive tissue contact via the K-factor method), system 10 may be designed such that a positive indication of tissue contact is output to a display and/or a clinician. Likewise, processing system 10 may, at times, sense a change in the K-factor corresponding to positive tissue contact despite not having sensed an impedance increase. Furthermore, it is contemplated that in some instances system 10 may simultaneously sense a change in the K-factor and an impedance increase, both of which provide a positive indication of tissue contact.

In addition or alternatively to any of the embodiments disclosed herein, improvements in the measurements of any electrical characteristic disclosed herein (e.g. impedance) may be achieved by utilizing a four-terminal sensing configuration among any of mapping electrodes 24 on electrode structure 20 (of which any number may be operated as sensing and/or current-carrying electrodes). In general, a four-terminal sensing configuration drives current through a pair of “current-carrying” electrodes and measures the voltage across a different pair of “sensing” electrodes.

One advantage of a four-terminal sensing configuration is that the measured impedance may not be sensitive to the impedance of the electrodes themselves. In a two-terminal sensing configuration, the measured impedance includes the surrounding medium and both electrodes. In contrast, a four-terminal sensing configuration measures voltage across electrodes through which the current is negligible. As a result, the measured impedance is that of the surrounding medium and is largely independent of the impedance of the electrode and its interface with the surrounding medium.

Additionally, in some instances, improvements in the measurements of any electrical characteristic disclosed herein (e.g. impedance) may be improved by utilizing a three-terminal sensing configuration among any of mapping electrodes 24 on electrode structure 20 (of which any number may be operated as sensing and/or current-carrying electrodes). Some examples of three-terminal sensing may be found in U.S. Pat. No. 8,449,535, the entirety of which is incorporated herein by reference. Further, in at least some instances, three-terminal sensing may be used instead of the four-terminal sensing configurations described herein, to the extent applicable.

It can be appreciated that four-terminal sensing may be incorporated and/or utilized by any combination of mapping electrodes 24 on electrode structure 20. Additionally, it is contemplated that any individual mapping electrode 24 on electrode structure 20 may operate as a sensing electrode or a current-carrying electrode. Additionally, as described above, system 10 may multiplex sensing configurations such that mapping electrodes 24 are both sensing and current carrying electrodes.

Furthermore, it is contemplated that sensing tissue contact utilizing the K-factor method, the impedance method or a combination of both can further incorporate four-terminal sensing as desired. For example, voltage values for the K-factor method may be obtained using four-terminal sensing. Likewise, impedance increase values for the impedance increase method may be obtained using four-terminal sensing. Additionally, either method may utilize four-terminal sensing in combination with any other method. For example, a “K-factor four terminal” method may be utilized simultaneously with the impedance increase method, which, in turn, may or may not incorporate four-terminal sensing. Additionally, an “impedance increase four terminal” method may be utilized simultaneously with the K-factor method, which, in turn, may or may not incorporate four-terminal sensing.

In some examples, mapping electrodes 24 may be operatively coupled to processor 32. Further, generated output from mapping electrodes 24 may be sent to processor 32 of system 10 for processing in one or more manners discussed herein and/or for processing in other manners. As stated, an electrical characteristic (e.g. impedance) and/or an output signal from an electrode pair may at least partially form the basis of a contact assessment.

Further, system 10 may be capable of processing or may be configured to process the electrical signals from mapping electrodes 24. Based, at least in part, on the processed output from mapping electrodes 24 processor 32 may generate an output to a display (not shown) for use by a physician or other user. In instances where an output is generated to a display and/or other instances, processor 32 may be operatively coupled to or otherwise in communication with the display. Illustratively, the display may include various static and/or dynamic information related to the use of system 10. In one example, the display may include one or more of an image of the target area, an anatomical shell, a map conveying tissue proximity achieved at locations on the anatomical shell, an electroanatomical map that incorporates tissue proximity information, an image of structure 20, and/or indicators conveying information corresponding to tissue proximity, which may be analyzed by the user and/or by a processor of system 10 to determine the existence and/or location of arrhythmia substrates within the heart, to determine the location of catheter 18 within the heart, and/or to make other determinations relating to use of catheter 18 and/or other elongated members.

System 10 may include an indicator in communication with processor 32. The indicator may be capable of providing an indication related to a feature of the output signals received from one or more of the electrodes of structure 20. In one example, an indication to the clinician about a characteristic of structure 20 and/or the myocardial tissue interacted with and/or being mapped may be provided on the display. In some cases, the indicator may provide a visual and/or audible indication to provide information concerning the characteristic of structure 20 and/or the myocardial tissue interacted with and/or being mapped. For example, system 10 may determine that a measured impedance corresponds to an impedance value of cardiac tissue and therefore may output a color indicator (e.g. green) to a display. The color indicator may allow a physician to more easily determine whether to apply ablative therapy to a given cardiac location. This is just an example. It is contemplated that a variety of indicators may be utilized by system 10.

In some embodiments, the processed output from mapping electrodes 24 may be used by processor 32 in ways that are not directly visible to the clinician. For example, processed information for contact assessment may be incorporated into algorithms for catheter localization, generation of anatomical shells and electroanatomical maps, or registration of images.

In some embodiments, the display may include an anatomical shell or an electroanatomical map that incorporates tissue proximity information. For example, regions of an anatomical shell where impedance values of cardiac tissue are measured may be more opaque than regions where impedance values of blood are measured. In other examples, an electroanatomical map displaying features such as voltage, activation time, dominant frequency, or the like may display an indicator (e.g. color, texture, pattern, etc.) in regions where impedance values of blood are measured. In both cases, the indication of regions where tissue contact may have occurred (or has likely occurred above a given probability or acceptability threshold) may guide the physician in moving the catheter and collecting measurements. Examples of anatomical shells and electroanatomical maps may be found in U.S. Patent Application Publication 20120184863, U.S. Patent Application Publication 20120184864 and U.S. Patent Application Publication 20120184865, the entirety of which is incorporated herein by reference.

In some examples, tissue proximity data may be collected for one or more mapping electrodes 24 on the structure 20 according to any of the processes and/or methods disclosed herein. Further, the collected parameter and/or tissue proximity values may be displayed on an anatomical shell and/or electroanatomical map as discussed above.

In other examples, tissue contact information may be used to mask portions of an anatomical shell and/or an electroanatomical map. Further, displayed (or masked) portions of the shell or map may correspond to a threshold confidence level of tissue contact. For example, masked portions may correspond to parameter values that are below a threshold confidence value.

As discussed above, the anatomical and/or electroanatomical map displaying (or masking) tissue contact locations may be manipulated by a clinician in order to generate more accurate diagnostic representations of an anatomical region (e.g. heart chamber).

The following documents are herein incorporated by reference: U.S. Patent Application Pub. US2008/0243214, U.S. Patent Application Pub. US2014/0058375, U.S. Patent Application Pub. US2013/0190747, U.S. Patent Application Pub. US2013/0060245, and U.S. Patent Application Pub. US2009/0171345.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electrophysiology medical device, comprising:
a catheter shaft including a distal end portion, wherein the distal end portion includes a sensing assembly having a plurality of mapping electrodes;
wherein the plurality of mapping electrodes includes at least one current-carrying electrode, a first sensing electrode and a second sensing electrode;
wherein the first sensing electrode is spaced from the current-carrying electrode a first distance;
wherein the second sensing electrode is spaced from the current-carrying electrode a second distance;
wherein the first distance is different from the second distance; and
a controller coupled to the plurality of mapping electrodes;
wherein the controller is capable of calculating a parameter based at least in part on the first and the second distances.

2. The medical device of claim 1, wherein the parameter indicates the proximity of the medical device to tissue.

3. The medical device of claim 1, wherein calculating the parameter includes sensing a first voltage potential between the first electrode and one or more return electrodes, and sensing a second voltage potential between the second electrode and one or more return electrodes.

4. The medical device of claim 3, wherein calculating the parameter includes solving at least one linear equation, and wherein the at least one linear equation includes the first distance, the second distance, the first voltage and the second voltage.

5. The medical device of claim 1, wherein the sensing assembly includes a plurality of splines, and wherein the plurality of electrodes are disposed on the plurality of splines.

6. The medical device of claim 1, wherein the sensing assembly includes a plurality of splines, and wherein the plurality of splines includes an outwardly facing surface, and wherein the plurality of electrodes are disposed on the outwardly facing surface.

7. The medical device of claim 1, wherein the sensing assembly includes a plurality of splines, and wherein the plurality of splines are arranged in a basket.

8. The medical device of claim 1, wherein the plurality of electrodes are each designed to sequentially and/or simultaneously operate in a sensing configuration and a current-carrying configuration.

9. The medical device of claim 1, further comprising displaying the parameter on a display.

10. The medical device of claim 9, wherein displaying the parameter includes displaying a confidence value corresponding to the parameter.

11. The medical device of claim 1, wherein the displaying the parameter on a display further includes displaying an anatomical shell and/or an electroanatomical map that indicates the proximity of one or more of the plurality of electrodes to tissue.

12. A system for sensing tissue contact, comprising:
a catheter shaft including a distal end portion, wherein the distal end portion includes a sensing assembly having a plurality of electrodes;
wherein the plurality of electrodes includes a current-carrying electrode, a first sensing electrode and a second sensing electrode;
wherein the first sensing electrode is positioned a first distance from the current-carrying electrode;
wherein the second sensing electrode is positioned a second distance from the current-carrying electrode;
wherein the first distance is different from the second distance;
a processor, wherein the processor is designed to:
simultaneously detect:
(a) a first parameter based at least in part on the first and second distances, and
(b) an impedance increase across at least one of the plurality of electrodes.

13. The system of claim 12, wherein the impedance increase is defined by a change in impedance by at least 100%.

14. The system of claim 12, wherein simultaneously detecting an impedance increase indicates that at least one of the plurality of electrodes is embedded in tissue.

15. The system of claim 12, wherein simultaneously detecting a first parameter based at least in part on the first and second distances includes sensing a first voltage potential between the first electrode and one or more return electrodes, and sensing a second voltage potential between the second electrode and the one or more return electrodes.

16. The system of claim 15, wherein simultaneously detecting a first parameter includes solving at least one linear equation, and wherein the at least one linear equation includes the first distance, the second distance, the first voltage and the second voltage.

17. The system in of claim 16, wherein simultaneously detecting an impedance increase includes measuring an impedance between the current-carrying electrode and one or more return electrodes.

18. An electrophysiology medical device, comprising:
a catheter shaft including a distal end portion;
a sensing assembly having a plurality of electrodes, wherein the plurality of electrodes includes four or more terminals;
wherein the four or more terminals includes one or more current-carrying electrodes and one or more sensing electrodes;
wherein the one or more current-carrying electrodes, the one or more sensing electrodes, or both includes a mapping electrode;
wherein the four or more terminals are designed to measure an electrical characteristic; and
a processor coupled to the sensing assembly.

19. The medical device of claim 18, wherein the electrical characteristic is a voltage, an impedance, or both.

20. The medical device of claim 18, wherein the electrical characteristic indicates the proximity of the medical device to tissue.
